(19) United States
(12) Patent Application Publication
(10) Pub. No.: US 2022/0061748 A1
(43) Pub. Date: Mar. 3, 2022

(54) MALE SEXUAL FUNCTION DETECTOR AND DETECTION SYSTEM

(71) Applicant: Yi-Ting LIN, Yunlin County (TW)

(72) Inventor: Yi-Ting LIN, Yunlin County (TW)

(21) Appl. No.: 17/317,919

(22) Filed: May 12, 2021

(30) Foreign Application Priority Data
Aug. 26, 2020 (TW) 109129204

(51) Int. Cl. A61B 5/00

(52) U.S. Cl. CPC A61B 5/4393; A61B 2562/221; A61B 2560/0214; A61B 5/002

(57) ABSTRACT

A male sexual function detector and a male sexual function detection system are provided. The male sexual function detector includes a housing, a detection element, a physical stimulation element, a waterproof cable, a bearing, a drive element, and a circuit board. The detection element, the physical stimulation element, the waterproof cable, the bearing, the drive element and the circuit board are disposed within the housing. The detection element includes a pressure sensor or a Doppler sensor. The physical stimulation element allows the bearing to rotate by the drive element, thereby driving the physical stimulation element. The detection element detects the sexual function. A detection signal is received by the circuit board to obtain the detection results. The male sexual function detection system includes a male sexual function detector, an electronic device, and a cloud database. The male sexual function detection system allows a subject to obtain detection results.

### CROSS REFERENCE TO RELATED APPLICATIONS

This application claims the priority of Taiwan Patent Application No. 109129204, filed on Aug. 26, 2020, titled “MALE SEXUAL FUNCTION DETECTOR AND DETECTION SYSTEM”, and the disclosure of which is incorporated herein by reference.

### FIELD OF INVENTION

The present disclosure relates to the technical field of a male sexual function detector, and particularly, to a male sexual function detector via physical stimulation. The present disclosure relates to a male sexual function detection system comprising the male sexual function detector.

### BACKGROUND OF INVENTION

Male sexual function mainly comprises two sexual responses: an erectile response and an ejaculation response. The sexual responses may be caused through psychological or physical stimulation. The psychological stimulation comprises visual stimulation, auditory stimulation or self-imaging. The physical stimulation is the physical stimulation of male genitals. In most cases, the psychological stimulation may cause the erectile response of the male genital, but the ejaculation response may not be caused under normal physiological conditions, while the physical stimulation may cause both the erectile response and the ejaculation response.

Under busy life and work stress, men often suffer from male sexual dysfunction. Therefore, literatures have reported that the prevalence of male sexual dysfunction is greater than 50%. It is obvious that there are many males suffering from male sexual dysfunction. In prior arts, a method for examining male sexual function mainly use the questionnaires, such as international index of erectile function and erection hardness score. The results of these questionnaires come from subjects' subjective cognition of their own sexual function, and the cognition is presented on a psychological scale. Since such questionnaires involve subjective judgments of subjects, it is impossible to accurately examine male sexual function through psychological scales.

In fact, male sexual function may also be examined by physical quantity detection. The physical quantity detection comprises the examination of blood flow in the penile artery at each time point, maximum erection length of the penis, sustained erection time, delay time before ejaculation, and a contraction pressure of urethral cavernous muscle during ejaculation. Therefore, more accurate examination results may be obtained through physical quantity detection as compared to the psychological scale of the questionnaires.

However, since sexual life is a private act, it is very difficult for medical staffs to obtain the results of these physical quantities. In addition, the male sexual function detector in the prior art performs examination by means of magnetic valve measurement, pressure measurement, or camera measurement. However, such male sexual function detector in the prior art may not provide a physical stimulus to the male genitals, not to mention the quantification and normalization of physical stimulation. Therefore, most of the subjects may only achieve the erectile response, but not ejaculate response. Thus, the male sexual function detector in the prior art may not examine the physical quantities related to ejaculation, and the prior art does not disclose a method for determining whether the obtained measured value is normal, either.

From the above, it is obvious that the male sexual function detector in the prior art may not quantify and standardize the sexual stimulation during men's sexual activity. As a result, medical staffs have to rely on the questionnaires to determine male sexual function, and the accurate examination results may not be obtained.

Therefore, the development of a male sexual function detector capable of examining the physical quantities to accurately examine male sexual function is an urgent problem in the art.

### SUMMARY OF INVENTION

In order to solve the above-mentioned problem that the male sexual function detector in the prior art may not accurately examine and physically quantify the male sexual function, one object of the present disclosure is to provide a male sexual function detector which may cause the penis to generate the erectile response and the ejaculation response by use of a physical stimulation element, and examine blood flow in the penile artery at each time point, maximum erection length of the penis, sustained erection time and delay time before ejaculation, and a contraction pressure of urethral cavernous muscle during ejaculation, and erection time before ejaculation by use of a detection element to obtain the accurate results of male sex function examination.

Another object of the present disclosure is to provide a male sexual function detection system, which may receive a wireless signal from a detection unit by an electronic device, and perform signal processing and noise reduction computing to obtain a processed electronic signal, and then analyze and rate the results by uploading the processed electronic signal to a cloud database, to obtain male sexual function examination results which may be sent back to the subjects.

To achieve the above-mentioned objects, the object of the present disclosure is to provide a male sexual function detector. The male sexual function detector comprises a first housing, a second housing, a third housing, a detection element, a waterproof cable socket, a physical stimulation element, a waterproof cable, a bearing, a drive assembly, and a circuit board. The first housing is a hollow housing and comprises an inlet end, a first accommodating space, and a first fastening. The inlet end is disposed at one end of the first housing. An inner peripheral wall of the first housing defines the first accommodating space. The first accommodating space communicates with the inlet end. The first fastening is disposed at another end of the first housing opposite to the inlet end.

The second housing is a hollow housing and comprises a second accommodating space, a second fastening, and a third fastening. An inner peripheral wall of the second housing defines the second accommodating space. The second fastening is disposed at one end of the second housing and matches the first fastening. The third fastening is disposed at another end of the second housing opposite to the second fastening.

The third housing comprises a third accommodating space, a bottom, and a fourth fastening. An inner peripheral wall and an inner bottom wall of the third housing define the third accommodating space. The fourth fastening is disposed at one end of the third housing opposite to the bottom, and the fourth fastening matches the third fastening.

The detection element is disposed inside the first housing. The detection element comprises a casing and a detector. The casing encapsulates the detector. The casing is a ring-shaped hollow structure and is disposed along the inner peripheral of the first housing. An outer peripheral wall of the casing abuts against the inner peripheral wall of the first housing. The detector comprises at least one pressure sensor or at least one Doppler sensor. The at least one pressure sensor or the at least one Doppler sensor is encapsulated in the casing.

The physical stimulation element is disposed inside the second housing. The physical stimulation element comprises a cup body and a silicone cup. The cup body comprises an open end, a cup pedestal, and a fourth accommodating space. The cup pedestal is disposed opposite to the open end of the cup body and is located on an outer bottom wall of the cup body. The cup pedestal has an inner peripheral wall and an outer peripheral wall. The inner peripheral wall of the cup pedestal defines a fifth accommodating space. The fourth accommodating space is formed between an inner peripheral wall and an inner bottom wall of the cup body.

The silicone cup is accommodated in the fourth accommodating space of the cup body. The silicone cup comprises an open end, a plurality of protrusions, and a six accommodating space. The open end of the silicone cup is adjacent to and communicates with the open end of the cup body. Each of the protrusions is disposed between the open end of the cup body and an inner bottom wall of the cup body. Each of the protrusions extends from an inner peripheral wall of the silicone cup toward a central axis of the silicone cup. A free end of each of the protrusions defines the six accommodating space.

The waterproof cable is disposed on the inner peripheral wall of the second housing and the inner peripheral wall of the third housing. The waterproof cable is electrically connected to the pressure sensor or the Doppler sensor.

The bearing is disposed inside the third housing. The bearing comprises a flange-typed linear bearing. The flange-typed linear bearing comprises a base and a projection. The base is perpendicularly connected to the projection of the flange-typed linear bearing. The base is accommodated and fixed in the fifth accommodating space of the cup pedestal. The projection of the flange-typed linear bearing comprises a axle hole inside the projection of the flange-typed linear. The axle hole of the projection of the flange-typed linear bearing extends from one end of the projection of the of the flange-typed linear bearing toward the base.

The drive assembly is disposed within the third accommodating space of the third housing. The drive assembly comprises a power supply and at least one circuit board. The power supply electrically is connected to the waterproof cable. The at least one motor is electrically connected to the power supply. The at least one motor is disposed adjacent to the physical stimulation element and opposite to the detection element. The at least one the motor comprises a rotation axis and the rotation axis penetrates the axel hole of the projection of the flange-typed linear bearing; and

The at least one circuit board comprises an integrated circuit. The at least one circuit board is disposed on the inner peripheral wall of the third housing and electrically connected to the waterproof cable.

In one embodiment, the casing further comprises a soft silicone pad and an elastic silicone layer. The soft silicone pad is a ring-shaped hollow structure and disposed around the inside of the first housing. An outer peripheral wall of the soft silicone pad abuts against the inner peripheral wall of the first housing. The elastic silicone layer is a ring-shaped hollow structure and disposed around an inside of the soft silicone pad. An outer peripheral wall of the elastic silicone layer abuts against an inner peripheral wall of the soft silicone pad. The at least one pressure sensor or the at least one Doppler sensor is embedded in the elastic silicone layer.

In one embodiment, the bearing further comprises a waterproof bearing disposed between the physical stimulation element and the at least one motor. The waterproof bearing comprises a axle hole, an inner bearing, an outer bearing and a waterproof seal. The axle hole of the waterproof bearing is formed in the inner bearing. The projection of the flange-typed linear bearing penetrates the axel hole of the waterproof bearing. The outer bearing is located outside the inner bearing and an outer peripheral wall of the outer bearings abuts against the inner peripheral wall of the third housing. The waterproof seal is disposed between the inner bearing and the outer bearing.

In one embodiment, the at least one motor comprises a rotary motor.

In one embodiment, the at least one motor further comprises a linear motor, and the linear motor is disposed adjacent to the rotary motor.

In one embodiment, the drive assembly further comprises two crankshafts, two linkages, and at least two slide rails. The two crankshafts are disposed oppositely and adjacent to the inner peripheral wall of the third housing. Each of the crankshafts is connected to the linear motor. The two linkages are disposed oppositely and adjacent to the inner peripheral wall of the third housing. An axis of each of the linkages is parallel to an axis of the male sexual function detector, and one end of each of the linkages is respectively connected to a corresponding one of the crankshafts. The at least two slide rails are disposed on the inner peripheral wall of the third housing, and each of the slide rails is respectively connected to the linear motor.

In one embodiment, the male sexual function detector further comprises a positioning member. The positioning element is disposed on an outer peripheral wall of the first housing.

In one embodiment, the male sexual function detector further comprises at least one through hole. The at least one through hole is defined by a bottom of the third housing.

In one embodiment, a number of the pressure sensor or the Doppler sensor is three. One of the pressure sensors or one of the Doppler sensors is disposed opposite to the positioning member of the first housing. Based on a baseline formed by the positioning member and the opposite pressure sensor or the Doppler sensor, each of the other two pressure sensors or each of the other two Doppler sensors is respectively disposed at the two sides of the baseline and located between the positioning member and the pressure sensor or the Doppler sensor which is opposite to the positioning member.

In one embodiment, the cup pedestal of the cup body further comprises a first hollow structure. The first hollow structure penetrates from an outer peripheral wall of the cup pedestal to the inner peripheral wall of the cup pedestal. An axis of the first hollow structure of the cup pedestal is perpendicular to the axis of the male sexual function detector, and the first hollow structure is penetrated by a fastening pin.

In one embodiment, the male sexual function detector further comprises at least one waterproof cable socket. The at least one waterproof cable socket is disposed on the inner peripheral wall of the first housing, the inner peripheral wall of the second housing, or a combination thereof. The at least one waterproof cable socket is electrically connected to the waterproof cable.

In one embodiment, each of the protrusions of the silicon cup is disposed opposite to each other.

In one embodiment, the waterproof seal of the waterproof bearing is a folding fan type-waterproof part.

In one embodiment, the base of the flange-typed linear bearing further comprises a second hollow structure. An axis of the second hollow structure of the base is perpendicular to the axis of the male sexual function detector. The second hollow structure communicates with the first hollow structure of the cup pedestal. The second hollow structure is penetrated by the fastening pin which matches the second hollow structure.

In one embodiment, a number of the at least one circuit board is two, and each circuit board is disposed on the inner peripheral wall of the third housing.

In one embodiment, the flange-typed linear bearing is a round flange-typed linear bearing.

In one embodiment, one of the at least one circuit board further comprises a Bluetooth module.

The present disclosure further provides a male sexual function detection system. The male sexual function detection system comprises the male sexual function detector described above, an electronic device, and a cloud database. The electronic device comprises a user interface and an integrated software element. The integrated software element is disposed in the electronic device. The integrated software element comprising a receiving unit, a transmission unit, and a processing unit. The receiving unit receives a wireless signal of a stimulus intensity command and receives electronic signals of penile hardness and pulsation transmitted by the circuit board of the male sexual function detector. The transmission unit transmits the wireless signal of the stimulus intensity command to the male sexual function detector. The processing unit performing signal processing and noise reduction computing on the electronic signals of penile hardness and pulsation to generate a processed electronic signal, wherein the processed electronic signal is transmitted to the user interface and the cloud database through the transmission unit.

The cloud database is a server and comprises an established initial database and a rating system. The cloud database receives the processed electronic signal transmitted by the transmission unit of the electronic device and generates a rating result. The rating result is transmitted back to the receiving unit of the electronic device via wireless transmission and displayed on the user interface.

In one embodiment, the processed electronic signal comprises a measured value of the penile hardness and a measured value of the pulsation.

In one embodiment, the measured value of the pulsation comprises an average stimulus intensity per unit time, an average duration of erection, an average pressure during erection, a maximum pressure during erection, total erection time before ejaculation, a contraction pressure of urethral cavernous muscle during ejaculation, erection time before ejaculation, or a combination thereof.

As mentioned above, the male sexual function detector of the present disclosure may provide physical stimulation of rotation and reciprocating twitch for a front end of the penis by use of the physical stimulation element and the drive assembly, so that the penis may generate the erectile response and the ejaculation response. In addition, the object of examining the blood flow in the penile artery at each time point, maximum erection length of the penis, sustained erection time, delay time before ejaculation, a contraction pressure of urethral cavernous muscle during ejaculation, and erection time before ejaculation to obtain the accurate results of male sex function examination at the same time may be achieved by the detector element. Moreover, the waterproof cable socket of the male sexual function detector of the present disclosure may be used to easily remove the first housing and the second housing to clean the physical stimulation element. Furthermore, the waterproof bearing of the male sexual function detector of the present disclosure may prevent liquid from infiltrating the rotary motor, the linear motor and the circuit board in the third housing.

In addition, the male sexual function detection system of the present disclosure may transmit the results examined by the male sexual function detector of the present disclosure to the cloud database through the electronic device, and perform normalization, analysis, and rating in the cloud database. Then the rating results may be sent back to the user interface of the electronic device, so that subjects may obtain accurate examination results and rating results timely.

### BRIEF DESCRIPTION OF DRAWINGS

In order to explain the technical solutions of the present disclosure more clearly, the following will briefly introduce the drawings used in the description of the embodiments or the related art. Obviously, the drawings described below are only some embodiments of the present disclosure. For those skilled in the art, other drawings can be obtained based on these drawings without making creative efforts.

FIG. 1 is a cross-sectional view of the male sexual function detector provided by one embodiment of the present disclosure.

FIG. 2 is a cross-sectional view along the A-A line in FIG. 1.

FIG. 3 is a cross-sectional view along the B-B line in FIG. 1.

FIG. 4 is a cross-sectional view along the C-C line in FIG. 1.

FIG. 5 is a cross-sectional view along the D-D line in FIG. 1.

FIG. 6 is a cross-sectional view along the E-E line in FIG. 1.

FIG. 7 is a cross-sectional view along the F-F line in FIG. 1.

FIG. 8 is a cross-sectional view along the G -G line in FIG. 1.

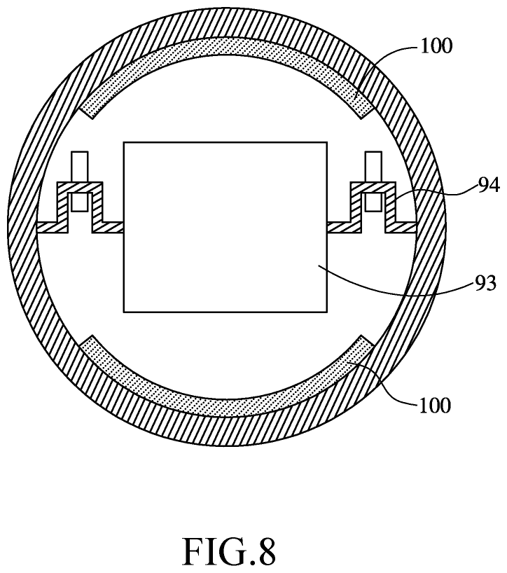

FIG. 9 is a schematic structural view of a male sexual function detection system provided by one embodiment of the present disclosure.

### DETAILED DESCRIPTION OF PREFERRED EMBODIMENTS

The following describes the embodiment of the present disclosure through specific examples. Those skilled in the field can understand other advantages and effects of the present disclosure from the content disclosed in the present specification. However, the exemplary embodiments disclosed in the present disclosure are merely for illustrative purposes and should not be construed as a limiting the scope of the present disclosure. In other words, the present disclosure can also be implemented or applied by other different specific embodiments, and various details in the present specification can also be modified and changed based on different viewpoints and applications without departing from the concept of the present disclosure.

The description of the following embodiments refers to the accompanying drawings to illustrate specific embodiments according to the present disclosure. The directional terms mentioned in the present disclosure, such as “upper”, “lower”, “both” sides”, “top”, “bottom”, “inner”, “outer”, “axial”, etc., refer only to the direction of the accompanying drawings. Therefore, the directional terms used in the present disclosure are used to describe and understand the present disclosure, rather than to limit scope of the present disclosure.

Unless otherwise described herein, the singular forms “a” and “the” used in the specification and the appended claims of the present disclosure comprise plural entities. Unless otherwise described herein, the term “or” used in the specification and the appended claims of the present disclosure comprises the meaning of “and/or”.

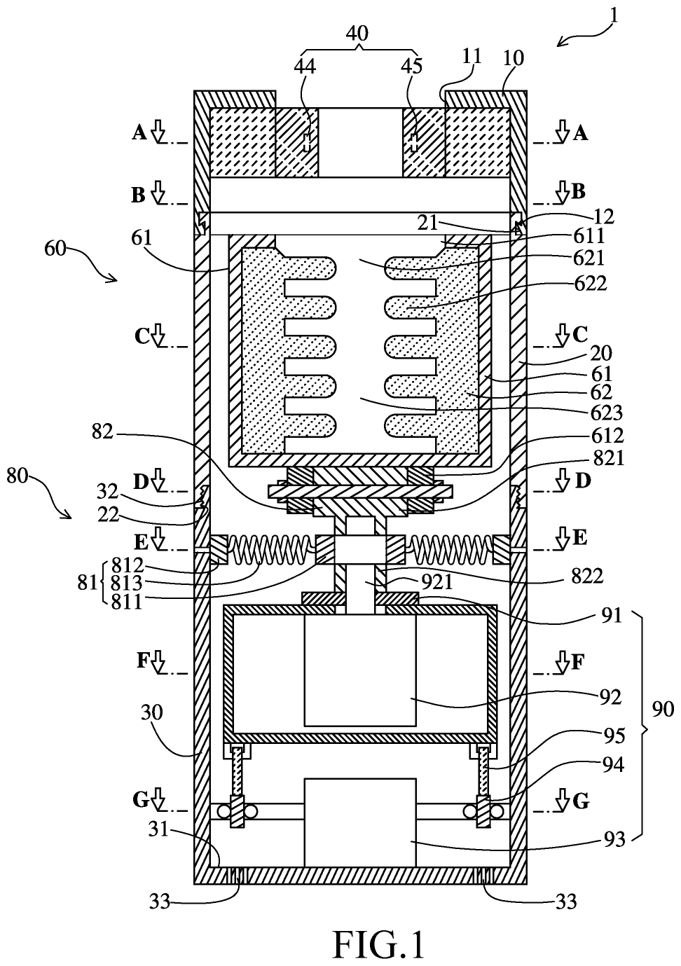

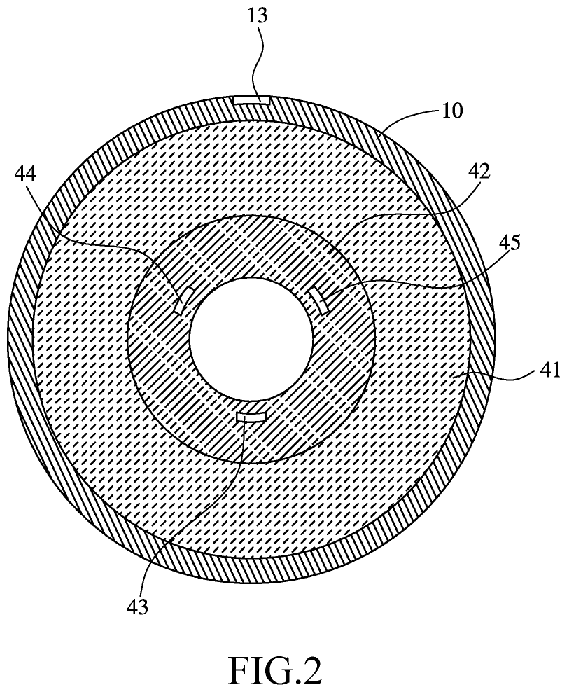

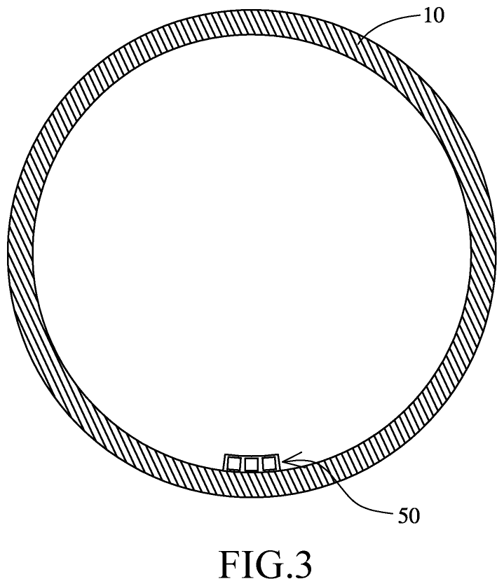

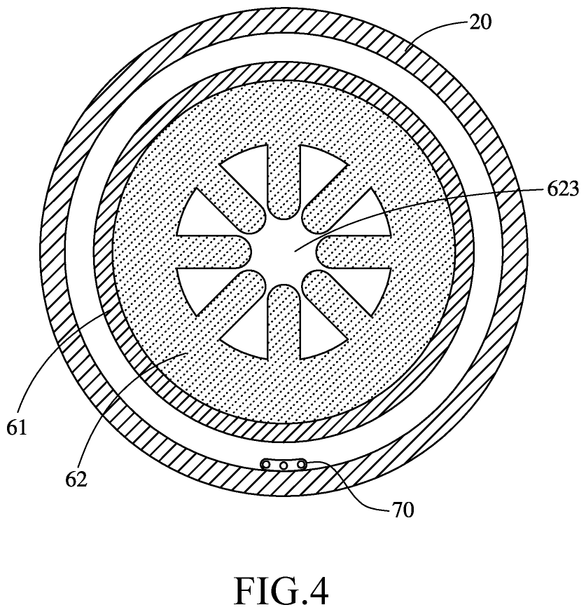

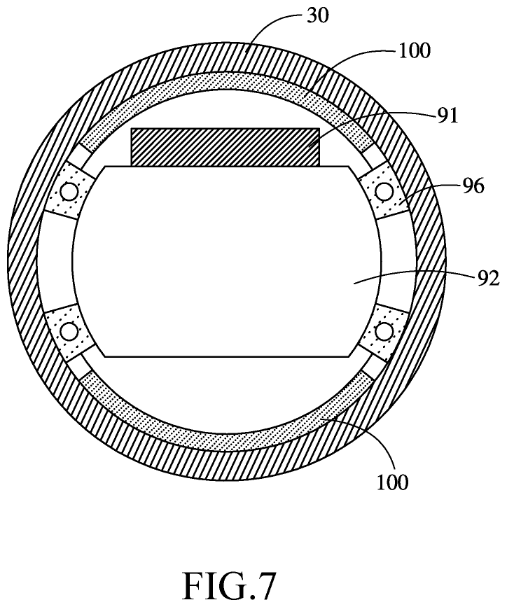

Referring to FIG. 1, FIG. 3, FIG. 4, and FIG. 7, the male sexual function detector 1 of the present disclosure comprises a first housing 10, a second housing 20, a third housing 30, a detection element 40, a waterproof cable socket 50, a physical stimulation element 60, a waterproof cable 70, a bearing 80, a drive assembly 90, and a circuit board 100. Referring to FIG. 1 and FIG. 2, the first housing 10 is a hollow housing and comprises an inlet end 11, a first accommodating space, a hook 12, and a positioning member 13. The inlet end 11 is disposed at one end of the first housing 10. An inner peripheral wall of the first housing 10 defines the first accommodating space, and the first accommodating space communicates with the inlet end of the first housing 10. The hook 12 is disposed at another end of the first housing 10 opposite to the inlet end 11. Referring to FIG. 2, the positioning member 13 is disposed on an outer peripheral wall of the first housing 10. The positioning member 13 indicates a direction of use of the male sexual function detector 1. In the present embodiment, the positioning member 13 is a mark directly above, which means when using the male sexual function detector 1, a subject has to have the positioning member 13 facing upward.

The second housing 20 is a hollow housing and comprises a second accommodating space, a slot 21, and an inner thread 22. An inner peripheral wall of the second housing 20 defines the second accommodating space, and the second accommodating space communicates with the first accommodating space of the first housing 10. The slot 21 is disposed at one end of the second housing 20, and the slot 21 matches the hook 12, so that the second housing 20 and the first housing 10 are detachably connected to each other. The inner thread 22 is disposed on another end of the second housing 20 opposite to the hook 12.

The third housing 30 comprises a third accommodating space, a bottom 31, an outer thread 32, and six through holes 33. An inner peripheral wall and an inner bottom wall of the third housing 30 define the third accommodating space. The outer thread 32 is disposed at another end of the third housing 30 opposite to the bottom 31, and the outer thread 32 is matched with the inner thread 22 of the second housing 20, allowing the third housing 30 and the second housing 20 to be detachably connected to each other. Each through hole 33 is defined by the bottom 31 of the third housing 30, allowing the third accommodating space to communicate with an outside of the third housing 30, to balance the air pressure inside and outside the male sexual function detector 1.

Referring to FIG. 1 and FIG. 2, the detection element 40 is disposed inside the first housing 10. The detection element 40 comprises a soft silicone pad 41, an elastic silicone layer 42, and three pressure sensors 43, 44, 45. The soft silicon pad 41 is a ring-shaped hollow structure and is disposed along the inner peripheral wall of the first housing 10, and an outer peripheral wall of the soft silicon pad 41 abuts against the inner peripheral wall of the first housing 10. The elastic silicone layer 42 is a ring-shaped hollow structure, and is disposed along an inner peripheral wall of the soft silicone pad 41. An outer peripheral wall of the elastic silicone layer 42 abuts against the inner peripheral wall of the soft silicone pad 41. The ring-shaped hollow structure of the elastic silicone layer 42 is an entrance of the subject's penis. Each of the pressure sensors 43, 44, 45 is respectively embedded in the elastic silicone layer 42, and the elastic properties of the elastic silicone layer 42 may provide pressure to the pressure sensors 43, 44, 45. The pressure sensor 43 is disposed opposite to the positioning member 13 of the first housing 10. In addition, based on a baseline formed by the positioning member 13 and the pressure sensor 43, each of the other two pressure sensors 44, 45 is respectively disposed at the two sides of the baseline and located between the positioning member 13 and the pressure sensor 43. When the male sexual function detector 1 of the present disclosure is used with the positioning member 12 facing upward, the pressure sensor 43 is used to examine the pressure generated by the urethral cavernous muscle of the penis on the pressure sensor 43 during the penile erection, and the pressure sensor 44 and the pressure sensor 45 are respectively used to examine the pressure generated by the cavernous bodies on both sides of the penis on the pressure sensor 44 and the pressure sensor 45 during the penile erection.

Referring to FIG. 1 and FIG. 3, the waterproof cable socket 50 is disposed on the inner peripheral wall of the first housing 10 and the inner peripheral wall of the second housing 20.

Referring to FIG. 1 and FIG. 4, the physical stimulation element 60 is detachably disposed inside the second housing 20. The physical stimulation element 60 comprises a cup body 61 and a silicone cup 62. The cup body 61 comprises an open end 611, a cup pedestal 612 and a fourth accommodating space. The open end 611 is adjacent to the slot 21 of the second housing 20 and the open end 611 communicates with the ring-shaped hollow structure of the elastic silicone layer 42. The cup pedestal 612 is disposed opposite to the open end 611 and is located on an outer bottom wall of the cup body 61. The cup pedestal 612 comprises an inner peripheral wall, an outer peripheral wall, and a first hollow structure. The first hollow structure penetrates from an outer peripheral wall of the cup pedestal 612 to the inner peripheral wall of the cup pedestal 612, and an axis of the first hollow structure of the cup pedestal 612 is perpendicular to an axis of the male sexual function detector 1 and may be penetrated by the fastening pin matched with the first hollow structure. The inner peripheral wall of the cup pedestal 612 defines a fifth accommodating space. The fourth accommodating space is formed between an inner peripheral wall and an inner bottom wall of the cup body 61.

The silicone cup 62 is disposed in the fourth accommodating space of the cup body 61, and the fourth accommodating space may accommodate the silicone cup 62. The silicone cup 62 comprises an open end 621, five sets of protrusions 622, and a sixth accommodating space 623. The open end 621 is adjacent to the open end 611 of the cup body 61 and communicates with the open end 611 of the cup body 61. Each set of protrusions 622 is equally spaced apart from each other between the open end 621 of the cup body 61 and an inner bottom wall of the cup body 61. Each set of protrusions 622 comprises eight protrusions 622 disposed opposite to each other, and each of the protrusions 622 extends from an inner peripheral wall of the silicone cup 62 toward a central axis of the silicone cup 62. A free end of each of the protrusions 622 defines a six accommodating space 623.

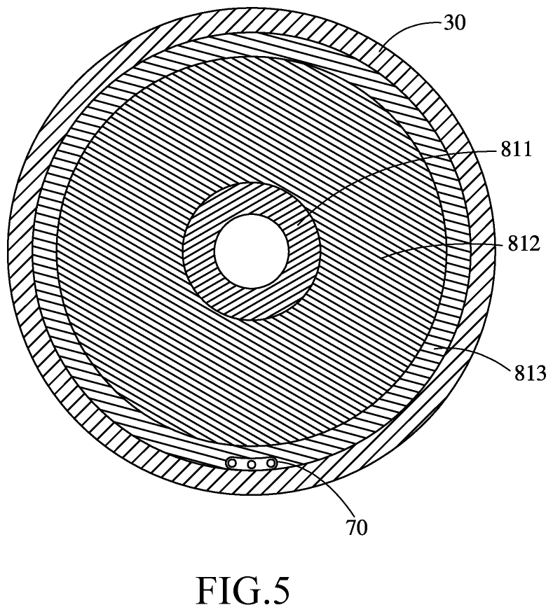

Referring to FIG. 1, FIG. 4, and FIG. 5, the waterproof cable 70 is disposed on the inner peripheral wall of the second housing 20 and the inner peripheral wall of the third housing 30. The waterproof cable 70 is electrically connected to the pressure sensor 43 and the waterproof cable socket 50 disposed on the first housing 10 and the second housing 20 to facilitate the removal of the first housing 10 and the second housing 20 for cleaning the physical stimulation element 60.

Referring to FIG. 1 and FIG. 5, the bearing 80 is disposed inside the third housing 30. The bearing 80 comprises a waterproof bearing 81 and a round flange-typed linear bearing 82. The waterproof bearing 81 comprises an axle hole, an inner bearing 811, an outer bearing 812, and a folding fan-typed waterproof part 813. The axle hole is formed inside the inner bearing 811. The outer bearing 812 is located outside the inner bearing 811 and an outer peripheral wall of the outer bearing 812 abuts against the inner peripheral wall of the third housing 30. The folding fan-typed waterproof part 813 is disposed between the inner bearing 811 and the outer bearing 812.

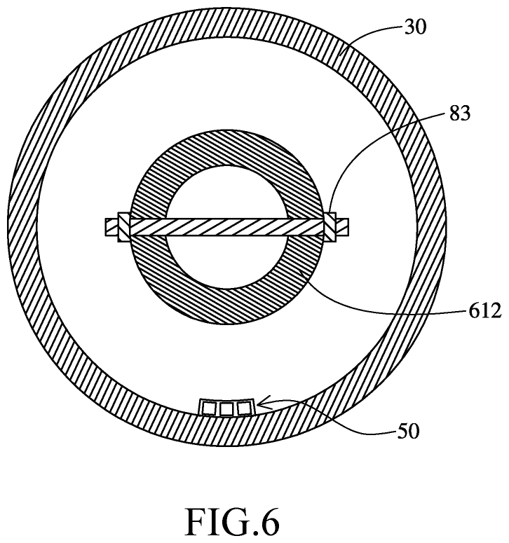

Referring to FIG. 1 and FIG. 6, the round flange-typed linear bearing 82 comprises a base 821 and a projection 822, the base 821 is perpendicularly connected to the projection 822, and the base 821 is accommodated in the fifth accommodating space of the base 612. The base 821 comprises a second hollow structure, and an axis of the second hollow structure of the base 821 is perpendicular to the axis of the male sexual function detector 1, and the second hollow structure communicates with the first hollow structure of the cup pedestal 612. The second hollow structure may be penetrated by the fastening pin matched the first hollow structure, and fixed by a screw nut 83, so that the cup pedestal 612 of the cup body 61 is stably combined with the base 821 of the round flange-typed linear bearing 82. The projection 822 of the round flange-typed linear bearing 82 comprises an axle hole. The axle hole of the projection 822 of the round flange-typed linear bearing 82 extends from one end of the projection 822 toward the base 821, and the projection 822 of the round flange-typed linear bearing 82 penetrates the axle hole of the waterproof bearing 81. An outer peripheral wall of the protruding part 822 and an inner wall of the axle hole of the waterproof bearing 81 are glued to avoid liquid infiltration.

Referring to FIG. 1 and FIG. 7, the drive assembly 90 is disposed in the third accommodating space of the third housing 30. the drive assembly 90 comprises a power supply 91, a rotary motor 92, and a linear motor 93, two crankshafts 94, two linkages 95, and two steel ball slide rails 96. The power supply 91 is electrically connected to the waterproof cable 70, the rotary motor 92, and the linear motor 93 to provide power to the rotary motor 92 and the linear motor 93. The rotary motor 92 is disposed adjacent to the waterproof bearing 81 and opposite to the physical stimulation element 60. The rotary motor 92 comprises a rotation axis 921. The rotation axis 921 penetrates the axle hole of the projection 822 of the round flange-typed linear bearing 82. When the power supply 91 supplies power, it may drive the round flange-typed linear bearing 82 to rotate, and at meanwhile drive the silicone cup 62 in the cup body 61 to rotate, so that the male sexual function detector 1 provides a rotary physical stimulation mode. In addition, the rotation speed of the rotary motor 92 may be adjusted through different input currents to generate physical stimulation mold with different intensities. In the present embodiment, based on different powers, five different physical stimulation intensities may be generated.

The linear motor 93 is a double-headed motor and is disposed on one side of the rotary motor 92 opposite to the waterproof bearing 81. Each of the crankshafts 94 is respectively disposed oppositely and located adjacent to the inner peripheral wall of the third housing 30 and connected to the linear motor 93. Each of the linkages 95 is disposed oppositely and located adjacent to the inner peripheral wall of the third housing 30. An axis of each of the linkages 95 is parallel to the axis of the male sexual function detector 1, and one end of each linkage 95 is respectively connected to a corresponding one of the crankshafts 94. Each of the steel ball slide rails 96 is disposed oppositely and adjacent to the inner peripheral wall of the second housing 20. One end of each steel ball slide rail 96 is connected to one end of each linkage 95 opposite to each crankshaft 94. Each of the steel ball slide rails 96 is disposed on the inner peripheral wall of the third housing 30 and is respectively connected to the linear motor 93. When the power supply 91 supplies power, the linear motor 93 may be driven to rotate the crankshafts 94 and drive the linkages 95 to push the steel ball slide rail 96, so that the silicone cup 62 in the cup body 61 may reciprocate along the axis of the male sexual function detector 1, and the male sexual function detector 1 provides a physical stimulation mode with reciprocating twitch. In addition, the rotation speed of the linear motor 93 may be adjusted through different input currents to generate physical stimulation mold with different intensities. In the present embodiment, based on different powers, five different physical stimulation intensities may be generated.

Each circuit board 100 is disposed on the inner peripheral wall of the third housing 30 and is electrically connected to the waterproof cable 70, so that the circuit board 100 is connected to the pressure sensor 43, and the currents inputting in the rotary motor 92 and the linear motor 93 are regulated by the circuit board 100. The circuit board 100 may comprises an integrated circuit and a Bluetooth module for wireless signal transmission.

When a subject uses the male sexual function detector 1 of the present disclosure, the subject may insert a penis 400 into the male sexual function detector 1 through the inlet end 11 of the first housing 10, so that the penis 400 is covered by the elastic silicone layer 42 of the detection element 40 and the silicone cup 62 of the physical stimulation element 60. The power supply 91 is then activated to make the silicone cup 62 of the physical stimulation element 60 rotate and reciprocate to stimulate a front end of the penis 400. After using, the subject may separate the first housing 10 from the second housing 20 by removing the hook 12 of the first housing 10 and the slot 21 of the second housing 20. The cup pedestal 612 may then be separated from the projection 822 of the round flange-typed linear bearing 82 by removing the fastening pin penetrating the first hollow structure of the cup pedestal 612 and the projection 822 of the round flange-typed linear bearing 82.

The second housing 20 may then separate from the third housing 30 by loosing the inner thread 22 22 of the second housing 20 and the outer thread 32 of the third housing 30 to clean the silicone cup 62 of the physical stimulation element 60. In addition, the silicone cup 62 of the physical stimulation element 60 is a consumable and should be replaced regularly to avoid elastic fatigue or deformation which may affect the test results. Furthermore, the waterproof bearing 81 of the present disclosure allows the third housing space of the third housing 30 where the rotary motor 92 and the linear motor 93 are disposed to achieve the waterproof effect, and prevent liquid from infiltrating the rotary motor 92, the linear motor 93 and the circuit board 100.

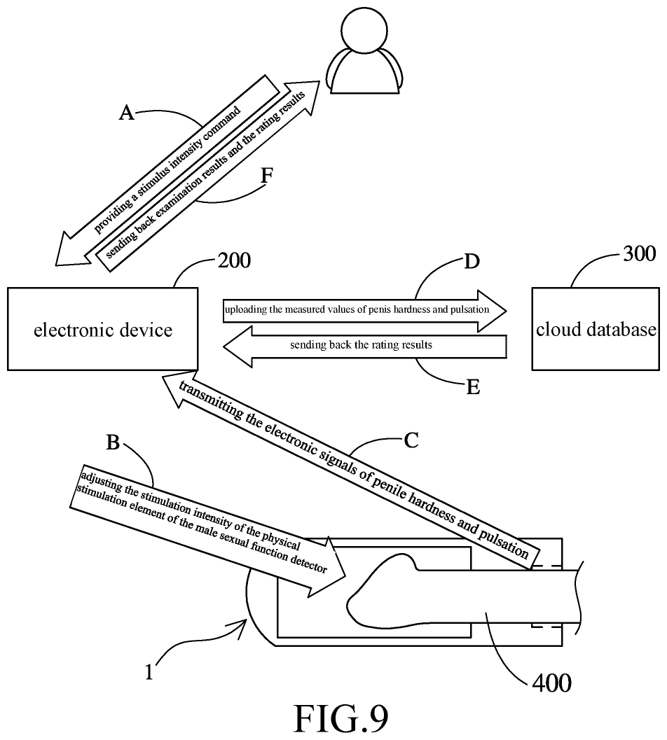

Referring to FIG. 1 and FIG. 9, the male sexual function detection system comprises a male sexual function detector 1, an electronic device 200, and a cloud database 300. The electronic device 200 comprises a user interface and an integrated software element. The integrated software element comprises a receiving unit, a transmission unit, and a processing unit. The receiving unit may receive a wireless signal transmitted by the Bluetooth module, and the transmission unit may transmit the wireless signal transmitted by the Bluetooth module. The processing unit may perform signal processing and noise reduction computing. The pressure values from the urethral cavernous muscle of the penis and the cavernous bodies on both sides of the penis measured by the pressure sensors 43, 44, 45 of the detection element 40 which are inputted over time are decomposed into trends, cycle changes, loop changes, and white noise. In the present embodiment, the trend is taken as a reference.

The cloud database 300 is a server. The server comprises an established initial database and a rating system. In the present embodiment, the establishment of the initial database comes from the recruitment of 30 self-identified subjects with normal sexual function, who are examined by the male sexual function detection system to obtain a normal pressure range for erection. Then, 100 men are randomly recruited, and the percentiles are established for the following measured values or ratios. The measured values comprise an average stimulus intensity per unit time, an average duration of erection, an average stimulation power ratio during erection, an average pressure during erection, a maximum pressure during erection, total erection time before ejaculation, an average stimulation power ratio during erection before ejaculation, and a contraction pressure of urethral cavernous muscle during ejaculation. Subjects using the male sexual function detector 1 of the present disclosure may obtain the percentile of all the measured values as a rating, and the measured values may be stored in the cloud database 300, so that the data may be expanded to make subsequent rating results more accurate.

Referring to FIG. 1 and FIG. 9, when the subject uses the male sexual function detection system of the present disclosure, the subject may put the penis 400 into the male sexual function detector 1. As shown by the arrow A in FIG. 9, the subject may input the desired physical stimulation mode and intensity generated by the physical stimulation element 60 to give a command of stimulation intensity. For example, the subject may choose a rotary physical stimulation mode or a reciprocating twitch physical stimulation mode, or a combination thereof. The subject may also choose the stimulation intensity from one to five level. As shown by the arrow B in FIG. 9, the wireless signal containing the command of the stimulation intensity is transmitted to the male sexual function detector 1 through the transmission unit of the integrated software element to adjust the stimulation strength of the physical stimulation element 60 of the male sexual function detector 1.

As shown by the arrow C in FIG. 9, after using the male sexual function detector 1 by the subject, the pressure sensors 43, 44, 45 of the detection element 40 of the male sexual function detector 1 may transmit the pressure values containing the electronic signals of the penile hardness and pulsation to the electronic device 200 through the Bluetooth module, followed by performing signal processing and noise reduction computing to obtain a processed electronic signal. The processed electronic signal comprises measured values of penile hardness and pulsation. The measured value of the pulsation comprises the examination results of an average stimulus intensity per unit time, an average duration of erection, an average pressure during erection, a maximum pressure during erection, total erection time before ejaculation, and a contraction pressure of urethral cavernous muscle during ejaculation.

The measured values of penile hardness and pulsation are transmitted to the user interface of the electronic device 200, so that the subject may obtain the examination results of the measured values of penile hardness and pulsation through the user interface. In addition, as shown by the arrow D in FIG. 9, the measured values of penile hardness and pulsation may also be uploaded to the cloud database 300 via the transmission unit of the electronic device 200, and the cloud database 300 may analyze and rate the processed electronic signals to generate the rating results. Finally, as shown by the arrow E in FIG. 9, the rating result is transmitted back to the electronic device 200 through wireless transmission. As shown by the arrow F in FIG. 9, the rating result is presented on the user interface of the electronic device 200, so that the subject may obtain the examination result and the rating result through the user interface of the electronic device 20.

Therefore, the male sexual function detector and the male sexual function detector system of the present disclosure may detect the physical quantity of male sexual function through the physical stimulation element 60, and upload the processed electronic signals to the user interface and the cloud database 300 through the transmission unit of the electronic device 200 for analysis and rating. The rating result may be transmitted back to the subjects to achieve the objective of objectively and accurately detecting male sexual function.

The above embodiments only illustrate the male sexual function detector and the male sexual function detector system of the present disclosure, and are not used to limit the present disclosure. A person skilled in the art can modify and change the above-mentioned embodiments without departing from the concept and scope of the present disclosure. Therefore, the claimed scope of the present disclosure should be determined based on the claims of patent disclosure.

What is claimed is:

1. A male sexual function detector, comprising:
a first housing being a hollow housing and comprising an inlet end, a first accommodating space, and a first fastening, wherein the inlet end is disposed at one end of the first housing; an inner peripheral wall of the first housing defines the first accommodating space; the first accommodating space communicates with the inlet end; and the first fastening is disposed at another end of the first housing opposite to the inlet end;
a second housing being a hollow housing and comprising a second accommodating space, a second fastening, and a third fastening, wherein an inner peripheral wall of the second housing defines the second accommodating space;
the second fastening is disposed at one end of the second housing and matches the first fastening; and the third fastening is disposed at another end of the second housing opposite to the second fastening;
a third housing comprising a third accommodating space, a bottom, and a fourth fastening, wherein an inner peripheral wall and an inner bottom wall of the third housing define the third accommodating space; and the fourth fastening is disposed at one end of the third housing opposite to the bottom, and the fourth fastening matches the third fastening;
a detection element disposed inside the first housing, wherein the detection element comprises a casing and a detector; the casing encapsulates the detector; the casing is a ring-shaped hollow structure and is disposed along the inner peripheral of the first housing; an outer peripheral wall of the casing abuts against the inner peripheral wall of the first housing; the detector comprises at least one pressure sensor or at least one Doppler sensor; and the at least one pressure sensor or the at least one Doppler sensor is encapsulated in the casing;
a physical stimulation element disposed inside the second housing, wherein the physical stimulation element comprises:
a cup body comprising an open end, a cup pedestal, and a fourth accommodating space, wherein the cup pedestal is disposed opposite to the open end of the cup body and is located on an outer bottom wall of the cup body; the cup pedestal has an inner peripheral wall and an outer peripheral wall; the inner peripheral wall of the cup pedestal defines a fifth accommodating space; and the fourth accommodating space is formed between an inner peripheral wall and an inner bottom wall of the cup body; and
a silicone cup being accommodated in the fourth accommodating space of the cup body, wherein the silicone cup comprises an open end, a plurality of protrusions, and a six accommodating space; the open end of the silicone cup is adjacent to and communicated with the open end of the cup body; each of the protrusions is disposed between the open end of the cup body and an inner bottom wall of the cup body, and each of the protrusions extends from an inner peripheral wall of the silicone cup toward a central axis of the silicone cup; and a free end of each of the protrusions defines the six accommodating space;
a waterproof cable disposed on the inner peripheral wall of the second housing and the inner peripheral wall of the third housing, wherein the waterproof cable is electrically connected to the pressure sensor or the Doppler sensor;
a bearing disposed inside the third housing, wherein the bearing comprises a flange-typed linear bearing; the flange-typed linear bearing comprises a base and a projection, the base is perpendicularly connected to the projection of the flange-typed linear bearing; the base is accommodated and fixed in the fifth accommodating space of the cup pedestal; the projection of the flange-typed linear bearing comprises a axle hole inside the projection of the flange-typed linear; and the axle hole of the projection of the flange-typed linear bearing extends from one end of the projection of the of the flange-typed linear bearing toward the base;
a drive assembly disposed within the third accommodating space of the third housing, wherein the drive assembly comprises:
a power supply electrically connected to the waterproof cable; and
at least one motor electrically connected to the power supply, wherein the at least one motor is disposed adjacent to the physical stimulation element and opposite to the detection element; the at least one the motor comprises a rotation axis; and the rotation axis penetrates the axel hole of the projection of the flange-typed linear bearing; and
at least one circuit board comprising an integrated circuit, wherein the at least one circuit board is disposed on the inner peripheral wall of the third housing and electrically connected to the waterproof cable.

2. The male sexual function detector according to claim 1, wherein the casing further comprises a soft silicone pad and an elastic silicone layer; the soft silicone pad is a ring-shaped hollow structure and disposed around the inside of the first housing; an outer peripheral wall of the soft silicone pad abuts against the inner peripheral wall of the first housing; the elastic silicone layer is a ring-shaped hollow structure and disposed around an inside of the soft silicone pad; an outer peripheral wall of the elastic silicone layer abuts against an inner peripheral wall of the soft silicone pad; and the at least one pressure sensor or the at least one Doppler sensor is embedded in the elastic silicone layer.

3. The male sexual function detector according to claim 1, wherein the bearing further comprises a waterproof bearing disposed between the physical stimulation element and the at least one motor; the waterproof bearing comprises a axle hole, an inner bearing, an outer bearing and a waterproof seal; the axle hole of the waterproof bearing is formed in the inner bearing, the projection of the flange-typed linear bearing penetrates the axel hole of the waterproof bearing; the outer bearing is located outside the inner bearing and an outer peripheral wall of the outer bearings abuts against the inner peripheral wall of the third housing; and the waterproof seal is disposed between the inner bearing and the outer bearing.

4. The male sexual function detector according to claim 1, wherein the at least one motor comprises a rotary motor.

5. The male sexual function detector according to claim 4, wherein the at least one motor further comprises a linear motor, and the linear motor is disposed adjacent to the rotary motor.

6. The male sexual function detector according to claim 5, wherein the drive assembly further comprises:
two crankshafts disposed oppositely and adjacent to the inner peripheral wall of the third housing, wherein each of the crankshafts is connected to the linear motor;
two linkages disposed oppositely and adjacent to the inner peripheral wall of the third housing, wherein an axis of each of the linkages is parallel to an axis of the male sexual function detector, and one end of each of the linkages is respectively connected to a corresponding one of the crankshafts; and
at least two slide rails disposed on the inner peripheral wall of the third housing, and each of the slide rails is respectively connected to the linear motor.

7. The male sexual function detector according to claim 1, further comprising a positioning member, wherein the positioning element is disposed on an outer peripheral wall of the first housing.

8. The male sexual function detector according to claim 1, further comprising at least one through hole, wherein the at least one through hole is defined by a bottom of the third housing.

9. The male sexual function detector according to claim 1, wherein a number of the pressure sensor or the Doppler sensor is three; one of the pressure sensors or one of the Doppler sensors is disposed opposite to the positioning member of the first housing; and based on a baseline formed by the positioning member and the opposite pressure sensor or the Doppler sensor, each of the other two pressure sensors or each of the other two Doppler sensors is respectively disposed at the two sides of the baseline and located between the positioning member and the pressure sensor or the Doppler sensor which is opposite to the positioning member.

10. The male sexual function detector according to claim 1, wherein the cup pedestal of the cup body further comprises a first hollow structure, wherein the first hollow structure penetrates from an outer peripheral wall of the cup pedestal to the inner peripheral wall of the cup pedestal; an axis of the first hollow structure of the cup pedestal is perpendicular to the axis of the male sexual function detector, and the first hollow structure is penetrated by a fastening pin.

11. The male sexual function detector according to claim 1, further comprising at least one waterproof cable socket, wherein the at least one waterproof cable socket is disposed on the inner peripheral wall of the first housing, the inner peripheral wall of the second housing, or a combination thereof, and the at least one waterproof cable socket is electrically connected to the waterproof cable.

12. The male sexual function detector according to claim 1, wherein each of the protrusions of the silicon cup is disposed opposite to each other.

13. The male sexual function detector according to claim 3, wherein the waterproof seal of the waterproof bearing is a folding fan type-waterproof part.

14. The male sexual function detector according to claim 1, wherein the base of the flange-typed linear bearing further comprises a second hollow structure, an axis of the second hollow structure of the base is perpendicular to the axis of the male sexual function detector; the second hollow structure communicates with the first hollow structure of the cup pedestal; and the second hollow structure is penetrated by the fastening pin which matches the second hollow structure.

15. The male sexual function detector according to claim 1, wherein a number of the at least one circuit board is two, and each circuit board is disposed on the inner peripheral wall of the third housing.

16. The male sexual function detector according to claim 1, wherein the flange-typed linear bearing is a round flange-typed linear bearing.

17. The male sexual function detector according to claim 1, wherein one of the at least one circuit board further comprises a Bluetooth module.

18. A male sexual function detection system, comprising a male sexual function detector according to claim 1, an electronic device, and a cloud database, wherein the electronic device comprises a user interface and an integrated software element, the integrated software element is disposed in the electronic device, and the integrated software element comprising:
a receiving unit receiving a wireless signal of a stimulus intensity command and receiving electronic signals of penile hardness and pulsation transmitted by the circuit board of the male sexual function detector;
a transmission unit transmitting the wireless signal of the stimulus intensity command to the male sexual function detector; and
a processing unit performing signal processing and noise reduction computing on the electronic signals of penile hardness and pulsation to generate a processed electronic signal, wherein the processed electronic signal is transmitted to the user interface and the cloud database through the transmission unit; and
wherein the cloud database is a server and comprises an established initial database and a rating system; the cloud database receives the processed electronic signal transmitted by the transmission unit of the electronic device and generates a rating result; and the rating result is transmitted back to the receiving unit of the electronic device via wireless transmission and displayed on the user interface.

19. The male sexual function detection system according to claim 18, wherein the processed electronic signal comprises a measured value of the penile hardness and a measured value of the pulsation.

20. The male sexual function detection system according to claim 19, wherein the measured value of the pulsation comprises an average stimulus intensity per unit time, an average duration of erection, an average pressure during erection, a maximum pressure during erection, total erection time before ejaculation, a contraction pressure of urethral cavernous muscle during ejaculation, erection time before ejaculation, or a combination thereof.
